(19) United States
(12) Patent Application Publication
(10) Pub. No.: US 2014/0094861 A1
(43) Pub. Date: Apr. 3, 2014

(54) SURGICAL LOCATOR

(71) Applicant: Smith & Nephew, Inc., Memphis, TN (US)

(72) Inventor: T. Wade Fallin, Hyde Park, UT (US)

(73) Assignee: Smith & Nephew, Inc., Memphis, TN (US)

(21) Appl. No.: 13/632,666

(22) Filed: Oct. 1, 2012

(51) Int. Cl. A61B 17/88

(52) U.S. Cl. 606/86 R

(57) ABSTRACT

A surgical locator and associated methods are presented. The surgical locator includes a shaft insertable into a surgical passage to maintain access to and indicate orientation of the surgical passage.

### FIELD OF THE INVENTION

The invention relates to devices to ease locating surgical incisions and other passages in soft and hard tissue during surgical procedures.

### BACKGROUND

During surgery, and especially during minimally invasive surgery, it can be difficult to maintain access to or orientation relative to incisions and other passages in soft and hard tissue. A need exists for devices and methods to aid in maintaining such access and orientation.

### SUMMARY

The present invention provides surgical locators and methods for making and using them.

In one aspect of the invention, a surgical locator includes an elongated shaft extending from a proximal end to a distal end and a barb formed at the distal end. The barb having a free state width corresponding to its maximum dimension measured perpendicular to the shaft when the barb is unconstrained in a free state condition and the barb being displaceable toward the shaft upon the application of a force toward the shaft and the barb being able to elastically return toward its free state condition after the force is removed.

In another aspect of the invention, the barb is sufficiently elastically deformable to permit its insertion into a passage smaller than its free state width and be elastically biased toward its free state condition to grip the passage.

In another aspect of the invention, the barb is sufficiently elastically and plastically deformable to permit its insertion into a passage smaller than its free state width and deform to a straightened configuration in which the free end of the barb is distal of the distal end of the shaft upon being withdrawn from the tunnel without the barb separating from the shaft.

In another aspect of the invention, a surgical method includes inserting a distal end of a surgical locator into a passage in a body tissue to engage a barb formed on the distal end of the surgical locator with the passage, the barb having a free state width wider than a portion of the passage such that the barb deforms elastically upon insertion into the passage and is elastically biased against the tunnel and subsequently referencing the surgical locator to locate the passage.

In another aspect of the invention, a surgical method includes placing a guide instrument relative to a surgical site, guiding a tunnel forming instrument with the guide instrument through soft tissue and into bone to form a passage through the soft tissue and in communication with the bone tunnel, guiding a surgical locator with the guide instrument through the passage and into the bone tunnel until a barb formed on the distal end of the surgical locator is engaged with the bone tunnel, removing the guide instrument from the surgical site while the surgical locator remains in the passage and bone tunnel, and subsequently referencing the surgical locator to locate the bone tunnel.

### BRIEF DESCRIPTION OF THE DRAWINGS

Various examples of the present invention will be discussed with reference to the appended drawings. These drawings depict only illustrative examples of the invention and are not to be considered limiting of its scope.

FIG. 1 is a side elevation view of the human foot illustrating anatomic reference planes;

FIG. 2 is a dorsal view of the metatarsus and proximal phalanx of the right second metatarsophalangeal joint of the human foot;

FIG. 3 is a medial view of the bones of FIG. 2;

FIG. 4 is a lateral view of the bones of FIG. 2;

FIG. 5 is a side elevation view of an illustrative example of a surgical locator according to the present invention;

FIG. 6 is a perspective view of an illustrative guide useable with the surgical locator of FIG. 5;

FIG. 7 is a side elevation view of the guide of FIG. 6 in use to create bone tunnels in underlying bones;

FIG. 8 is a top plan view of the guide of FIG. 6 in use to create bone tunnels in underlying bones;

FIG. 9 is a side elevation view of the surgical locator of FIG. 5 in use with the guide of FIG. 6 to locate an incision and bone tunnel;

FIG. 10 is a side elevation view of the surgical locator of FIG. 5 in use to locate an incision and bone tunnel after the guide of FIG. 6 has been removed; and

FIGS. 11 and 12 are side elevation views illustrating the removal of the surgical locator of FIG. 5.

### DESCRIPTION OF THE ILLUSTRATIVE EXAMPLES

The following illustrative examples illustrate surgical locators and methods for making and using them. Surgical locators according to the present invention may be used in conjunction with any surgical procedure but the illustrative examples are shown in a size and form most suitable for procedures involving the hand and foot. In particular, the illustrative examples depict their use around metatarsophalangeal (MTP) joints of the human foot. The illustrative surgical locators are also suitable for use around metacarpophalangeal (MCP) joints of the human hand. The hand and foot have a similar structure. Each has a volar aspect. In the hand the volar, or palmar, aspect includes the palm of the hand and is the gripping side of the hand. In the foot the volar, or plantar, aspect is the sole of the foot and is the ground contacting surface during normal walking. Both the hand and foot have a dorsal aspect opposite the volar aspect. Both the hand and foot include long bones referred to as metapodial bones. In the hand, the metapodial bones may also be referred to as metacarpal bones. In the foot, the metapodial bones may also be referred to as metatarsal bones. Both the hand and foot include a plurality of phalanges that are the bones of the digits, i.e. the fingers and toes. In both the hand and foot, each of the most proximal phalanges forms a joint with a corresponding metapodial bone. This joint includes a volar plate or band of connective tissue on the volar side of the joint. The joint also includes collateral ligaments on the medial and lateral sides of the joint. A transverse ligament connects the heads of the metapodial bones. In the hand the joint is typically referred to as the metacarpophalangeal joint having a palmar plate on the palmar side, collateral ligaments medially and laterally, and a transverse ligament connecting the metacarpals. In the foot the joint is typically referred to as the metatarsophalangeal joint having a plantar plate on the plantar side, collateral ligaments medially and laterally including proper collateral ligaments and accessory collateral ligaments, and a transverse ligament also known as the transverse metatarsal ligament.

For convenience, the illustrative examples depict the use of instruments and techniques according to the present invention to locate incisions and passages in hard and soft tissue around the metatarsophalangeal (MTP) joints of the human foot. The illustrative instruments and techniques are also suitable for use around the metacarpophalangeal (MCP) joints of the human hand and at other surgical sites. To better orient the reader, the MTP joint and basic anatomic references are explained in more detail below.

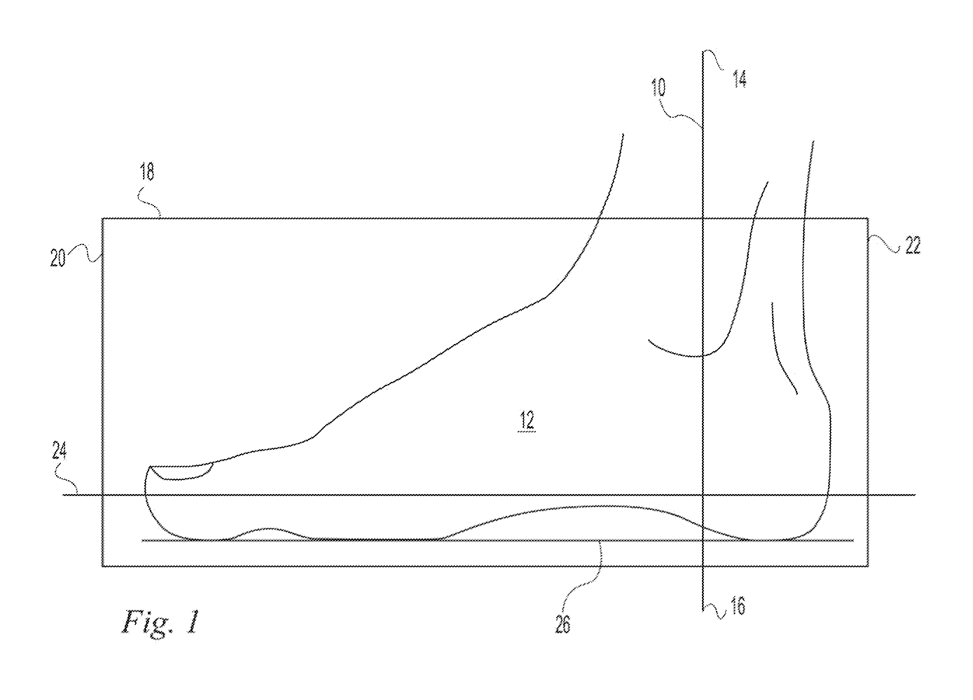

FIG. 1 illustrates the anatomic planes of the foot that are used for reference in this application. The coronal plane 10 extends from the medial aspect 12 to the lateral aspect of the foot and from dorsal 14 to plantar 16 and divides the foot between the toes and heel. The sagittal plane 18 extends anterior 20 to posterior 22 and dorsal 14 to plantar 16 and divides the foot into medial and lateral halves. The transverse plane 24 extends anterior 20 to posterior 22 and medial to lateral parallel to the floor 26.

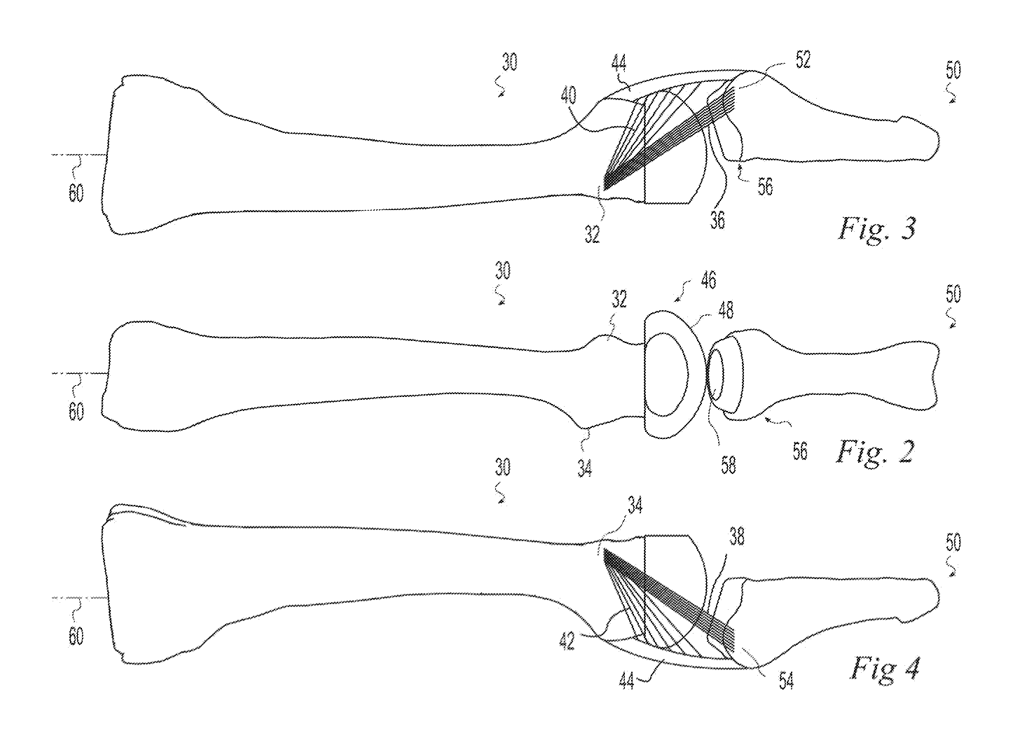

FIGS. 2-4 illustrate the metatarsus 30 and proximal phalanx 50 of the second MTP joint of the right foot. The medial and lateral epicondyles 32, 34, located on the medial-dorsal and lateral-dorsal aspects of the metatarsus 30 respectively, are the origins of the medial and lateral proper collateral ligaments (PCLs) 36, 38 and the medial and lateral accessory collateral ligaments (ACLs) 40, 42 of the MTP joint. The medial PCL inserts at the medial-plantar aspect 52 and the lateral PCL inserts at the lateral-plantar aspect 54 of the proximal phalanx 50. The ACLs fan out and insert into the plantar plate 44. The metatarsus includes a metatarsal head 46 having an articular surface 48 and the proximal phalanx includes a phalangeal head 56 having an articular surface 58. The metatarsus 30 further includes a longitudinal axis 60 extending lengthwise down the center of the bone.

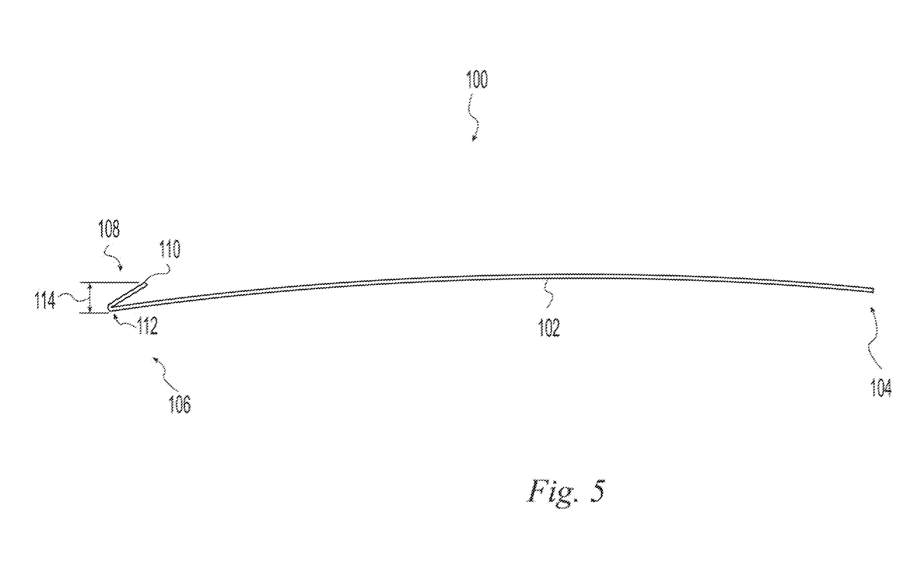

FIG. 5 illustrates an exemplary surgical locator 100 according to the present invention. The surgical locator 100 includes an elongated shaft 102 extending from a proximal end 104 to a distal end 106. A barb 108 is formed at the distal end. In the illustrative example of FIG. 5, the barb 108 is connected to the distal end 106 and extends proximally from the distal end terminating at an end 110. The barb has a free state width 114 corresponding to its widest dimension measured perpendicular to the shaft 102 when the barb is unrestrained. In the illustrative example of FIG. 5, this widest dimension occurs at the barb end 110. Preferably, the free state width 114 of the barb is greater than the width of a passage into which the barb is inserted to that the barb is displaced toward the shaft 102 and is elastically biased outwardly against the side of the passage to resist falling out of the passage or being inadvertently pulled from the passage. The surgical locator may be formed of any suitable material including, but not limited to, metals and polymers. Preferably the surgical locator 100 is formed of a material that is elastically deformable over a relatively large displacement such that it tends to return to its free state configuration. Likewise, it is preferable that the material properties permit the barb to be straightened, i.e. bent so that the end 110 points distally, at least temporarily, when it is pulled manually by a user from a narrow tunnel such as a bone tunnel. Suitable materials include materials that may be formed into the desired barbed configuration. For example, metal wire may be bent to form a barb at an end. In another example, a superelastic filament such as a nitinol wire may have a barb formed by first heating an end of the filament to transform the nitinol into its austenite phase and then bending it to form a barb and subsequently cooling it to the martensite phase. In another example, a monofilament polymer strand may be bent to form a barb. For example, a nylon monofilament may be bent and crimped to lock in the barb shape. Alternatively, the monofilament may be heated before or after bending to facilitate plastic deformation and setting of the barb shape. The heating may be localized at the bend 112.

Preferably the barb permits sufficient elastic and plastic deformation in use that with the application of sufficient force to withdraw it from a tunnel it is able to straighten out without breaking.

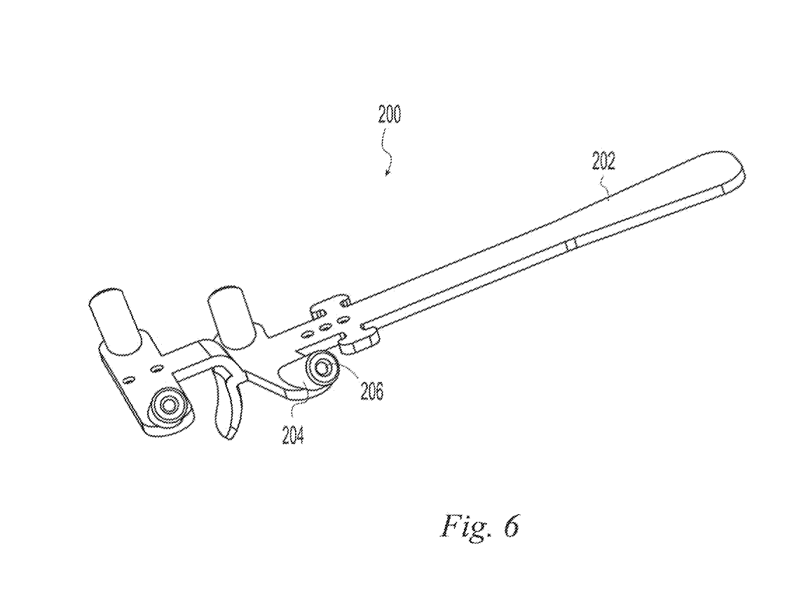

FIG. 6 depicts an illustrative example of a guide 200 useable with the surgical locator 100. The illustrative drill guide includes a handle 202 and one or more tubular guides 204 having a guide bore 206 able to guide a forming tool such as a drill, punch, pin, or the like to form a passage through soft and/or hard tissue. The illustrative guide 200 is just one of many configurations that may be used with the surgical locator 100 and is not limiting of the scope of the invention.

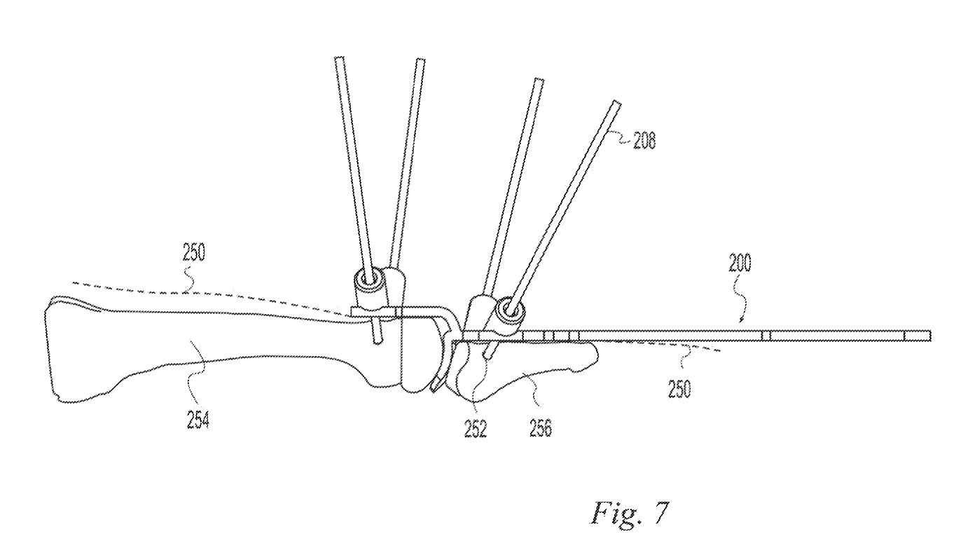

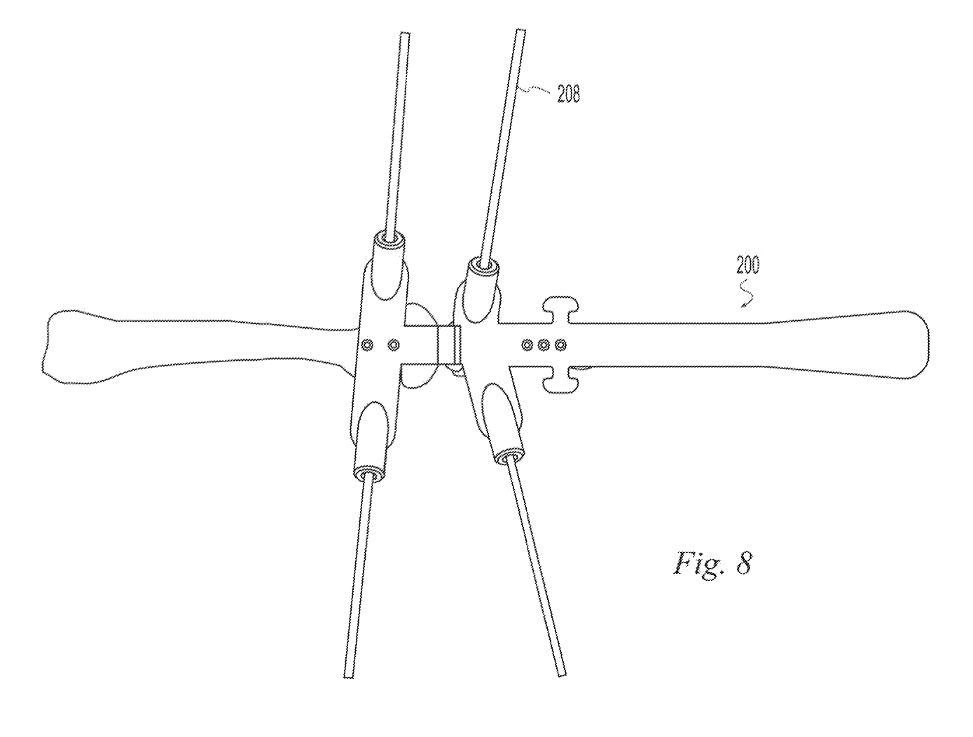

FIGS. 7-12 depict an illustrative surgical method utilizing the surgical locator of FIG. 5 in use to locate surgical incisions and other passages in soft and hard tissue. In FIGS. 7 and 8 the guide 200 is placed over a bone and a pin 208 guided through the guide bore 206 to form a passageway through the skin 250, through underlying soft tissue, and into the bone to form a bone tunnel 252. In the illustrative example of FIGS. 7-12 the underlying bone includes a metatarsal bone 254 and a proximal phalanx 256 adjacent an MTP joint and two pins 208 have been inserted through the soft tissue and into each bone.

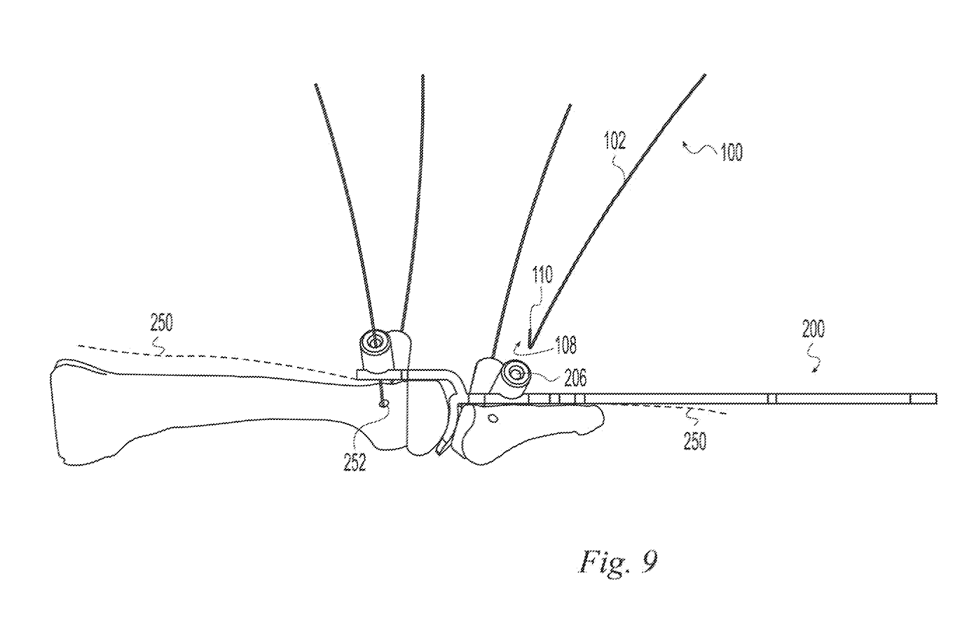

Referring to FIG. 9, the pins 208 has been removed and surgical locators 100 inserted through the guide 200 and through the skin 250, underlying soft tissues, and into the bone tunnels 252. One surgical locator 100 is shown as it is being inserted into the guide 200. The barb 108 bends back elastically toward the shaft 102 as it is inserted into the guide bore 206, through the skin 250 and underlying soft tissue, and into the bone tunnel 252. The barb 108 is biased back toward its free state configuration due to the elasticity of the surgical locator material. Such biasing causes the end 110 to engage the bone tunnel wall and resist removal of the surgical locator 100 from the bone tunnel 252.

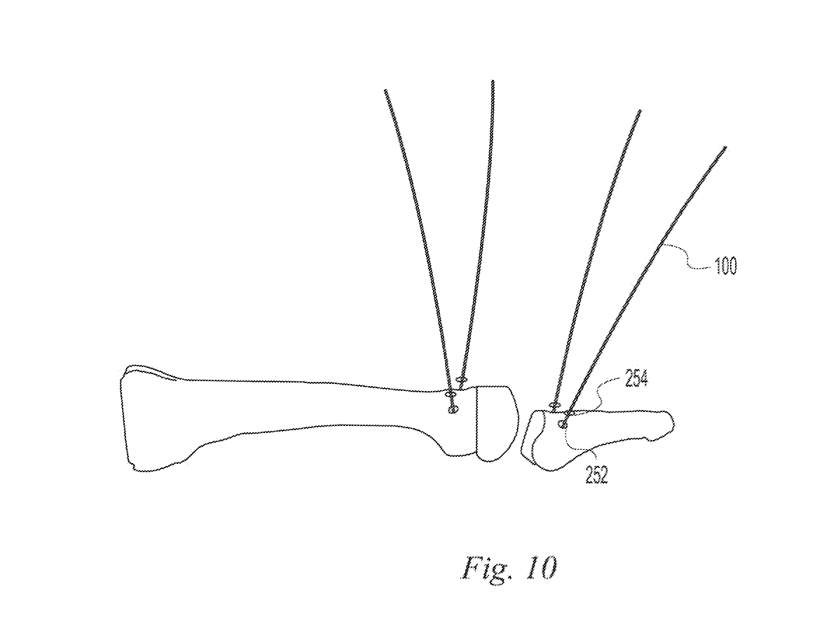

Referring to FIG. 10, the guide 200 has been removed and the surgical locators 100 left in place to locate the passage through the skin, other soft tissue, and into the bone tunnel. As the surgeon has need to access the passage or bone tunnel, he may follow the shaft of the surgical locator. For example, a device such as an instrument or implant may be guided to the bone tunnel 252 by sliding the instrument over or alongside the surgical locater 100 through the skin incision 254, through underlying soft tissue, and to the bone tunnel 252 entrance. Likewise the surgeon may use blunt dissection along the passage marked by the surgical locator until he can directly visualize, e.g. the bone tunnel, and then engage the bone tunnel as desired to perform the procedure. As necessary or desired, the surgical locator 100 may be removed by applying a proximally directed axial force sufficient to overcome the engagement of the barb 108 with the bone tunnel either by overcoming the friction force or straightening the barb so it no longer resists removal.

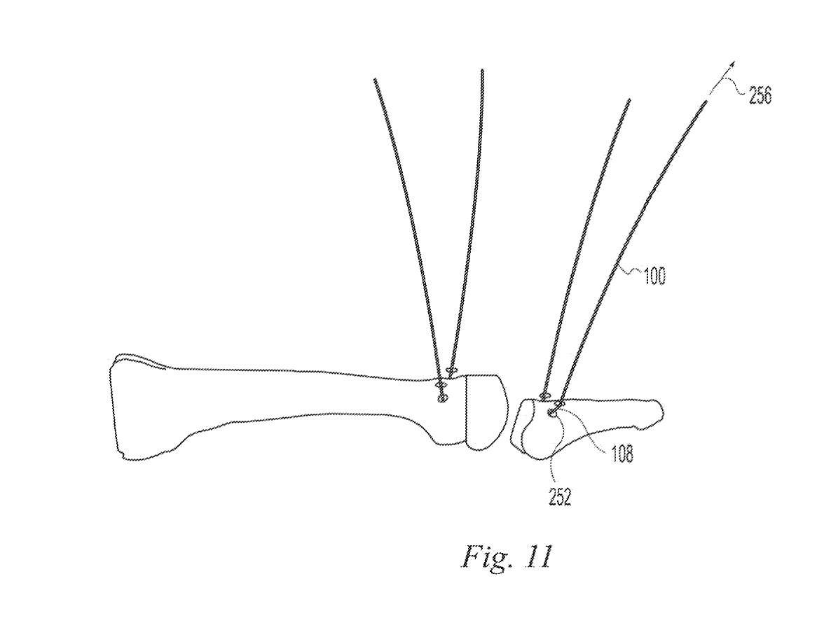

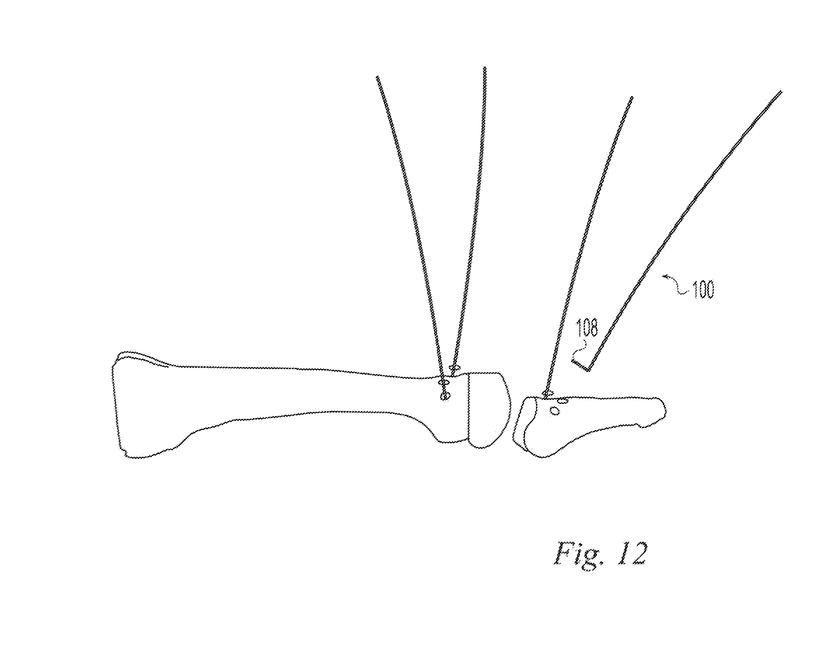

Referring to FIGS. 11 and 12, a surgical locator 100 is shown as it is being removed from the bone tunnel 252 by applying a proximally directed axial force 256 sufficient to straighten the barb 108. In FIG. 12, the surgical locator 100 is shown fully removed and with the barb 108 partially elastically recovered to its free state condition. If desired, the surgical locator may be reinserted, for example, by sliding it along an instrument inserted in the bone tunnel until it is adjacent the bone tunnel entrance and, after withdrawing the instrument, inserting the surgical locator.

The illustrative examples have depicted a surgical locator constructed and used for surgical procedures involving an MTP joint of a human foot. The surgical locator and methods of the present invention are suitable for procedures at other locations within a patient's body including, but not limited to, the MCP joints of the human hand.

What is claimed is:

1. A surgical locator comprising:
an elongated shaft extending from a proximal end to a distal end; and
a barb formed at the distal end, the barb extending proximally from the distal end and terminating at a free end spaced from the shaft, the barb having a free state width corresponding to its maximum dimension measured perpendicular to the shaft when the barb is unconstrained in a free state condition, the barb being displaceable toward the shaft upon the application of a force toward the shaft and the barb being able to elastically return toward its free state condition after the force is removed.

2. The surgical locator of claim 1 wherein the barb is made of a flexible monofilament.

3. The surgical locator of claim 1 wherein the shaft and barb are made of a continuous piece of flexible monofilament

4. The surgical locator of claim 3 wherein the material is heat processed to set the barb.

5. The surgical locator of claim 4 wherein the material is selected from the group consisting of superelastic materials and heat settable polymers.

6. The surgical locator of claim 5 wherein the material is selected from the group consisting of nitinol and nylon.

7. The surgical locator of claim 1 wherein the barb is sufficiently elastically deformable to permit its insertion into a passage smaller than its free state width and be elastically biased toward its free state condition to grip the passage.

8. The surgical locator of claim 1 wherein the barb is sufficiently elastically and plastically deformable to permit its insertion into a passage smaller than its free state width and deform to a straitened configuration in which the free end of the barb is distal of the distal end of the shaft upon being withdrawn from the tunnel without the barb separating from the shaft.

9. A surgical method comprising:
inserting a distal end of a surgical locator into a passage in a body tissue to engage a barb formed on the distal end of the surgical locator with the passage, the barb having a free state width wider than a portion of the passage such that the barb deforms elastically upon insertion into the passage and is elastically biased against the passage; and
subsequently referencing the surgical locator to locate the passage.

10. The surgical method of claim 9 wherein inserting a distal end of a surgical locator into a passage includes guiding the surgical locator through a guide instrument having a guide feature aligned with the passage.

11. The surgical method of claim 9 further comprising before the step of inserting a distal end of a surgical locator into a passage:
placing the guide instrument relative to a surgical site;
guiding a tunnel forming instrument with the guide to form a bone tunnel.

12. The surgical method of claim 11 further comprising:
guiding the tunnel forming instrument through skin, soft tissue, and into bone to form the bone tunnel.

13. The surgical method of claim 12 wherein the bone includes bone adjacent the metatarsophalangeal joint of a human foot and the method further comprises:
guiding the tunnel forming instrument to form a first bone tunnel in a metatarsal bone and a second bone tunnel in a proximal phalanx; and
inserting a surgical locator into each of the metatarsal bone tunnel and the proximal phalanx bone tunnel.

14. The surgical method of claim 9 further comprising after the step of subsequently referencing the surgical locator:
pulling the surgical locator proximally to remove it from the passage.

15. The surgical method of claim 14 wherein pulling the surgical locator causes the barb to straighten.

16. A surgical method comprising:
placing a guide instrument relative to a surgical site;
guiding a tunnel forming instrument with the guide instrument through soft tissue to form a soft tissue passage and into bone to form a bone tunnel, the passage through the soft tissue being in communication with the bone tunnel;
guiding a surgical locator with the guide instrument through the passage and into the bone tunnel until a barb formed on the distal end of the surgical locator is engaged with the bone tunnel;
removing the guide instrument from the surgical site while the surgical locator remains in the passage and bone tunnel; and
subsequently referencing the surgical locator to locate the bone tunnel.

17. The surgical method of claim 16 wherein the bone includes bone adjacent the metatarsophalangeal joint of a human foot and wherein the step of guiding a tunnel forming instrument includes guiding the tunnel forming instrument to form a first bone tunnel in a metatarsal bone and a second bone tunnel in a proximal phalanx and wherein the step of guiding a surgical locator includes guiding a first surgical locator into the first bone tunnel and a second surgical locator into the second bone tunnel.

18. The surgical method of claim 16 further comprising:
pulling the surgical locator proximally to remove it from the passage.

19. The surgical method of claim 18 wherein pulling the surgical locator causes the barb to straighten.

20. The surgical method of claim 16 wherein subsequently referencing the surgical locator comprises engaging a device with the shaft of the surgical locator and sliding the device along the shaft to locate the bone tunnel.
